(19) United States
(12) Patent Application Publication
(10) Pub. No.: US 2013/0131177 A1
(43) Pub. Date: May 23, 2013

(54) USE OF A DERMATOLOGICAL COMPOSITION COMPRISING A COMBINATION OF ADAPALENE AND BENZOYL PEROXIDE WHICH IS INTENDED FOR THE TREATMENT OF ACNE IN NON-CAUCASIAN POPULATION WITH DECREASE OF POST-INFLAMMATORY HYPERPIGMENTATION

(71) Applicants: Jean-Charles Dhuin, Nice (FR); Nabil Kerrouche, Le Rouret (FR); Pascale Soto, Antibes (FR)

(72) Inventors: Jean-Charles Dhuin, Nice (FR); Nabil Kerrouche, Le Rouret (FR); Pascale Soto, Antibes (FR)

(73) Assignee: GALDERMA RESEARCH & DEVELOPMENT, Biot (FR)

(21) Appl. No.: 13/578,170

(22) PCT Filed: Feb. 3, 2011
(86) PCT No.: PCT/EP2011/051580

§ 371 Date: Oct. 25, 2012

Related U.S. Application Data
(60) Provisional application No. 61/302,582, filed on Feb. 9, 2010.

(51) Int. Cl. A61K 31/327; A61K 31/192

(52) U.S. Cl. CPC A61K 31/327; A61K 31/192
USPC 514/569

(57) ABSTRACT

A dermatological composition including a combination of Adapalene and benzoyl peroxide is described. The composition is intended for the treatment of acne in the non-Caucasian population with decreased post-inflammatory hyperpigmentation, keloid scarring and acne hyperpigmented macules and cystic lesions.

The present invention relates to a dermatological composition comprising a combination of Adapalene and benzoyl peroxide which is intended for the treatment of acne in non-Caucasian population with decrease of post-inflammatory hyperpigmentation, keloid scarring and acne hyperpigmented macules and cystic lesions.

Acne vulgaris, also known as polymorphic acne is most common. It includes four stages, but passage through all stages is not obligatory:
Stage 1 corresponds to acne with Comedones characterized by a large number of open comedones and/or closed, and microcysts.
Stage 2, or papulopustular acne is mild to moderate. It is characterized by the presence of open comedones and/or closed, microcysts, but also red papules and pustules. It mainly affects the face and leaves little scarring.
Stage 3 or papulocomédonienne acne is more severe and extends to the back, chest and shoulders. It is accompanied by a greater number of scars.

Acne affects individuals of all races and ethnicities. The pathogenesis of acne is multifactorial, and the same factors are probably involved across the spectrum of skin types: sebaceous follicle obstruction, excessive sebum production due to hormonal stimulation of sebaceous glands, proliferation of Propionibacterium acnes, which produces chemotactic factors and proinflammatory mediators that, in turn, generate an inflammatory response, followed by follicular rupture and extension of inflammation into the dermis, resulting in the formation of inflammatory lesions.

The overall goal of acne management in all patients is to select treatment that effectively addresses as many of pathogenic factors as possible while minimizing side effects. Using multiple agents at the same time during treatment (concomitant therapy) has been recommended as a rational means to achieve this goal. Acne therapy in skin of color (high melanin content) presents unique challenges due to differences relating to acne sequelae in these skin types, especially the presence or risk of post-inflammatory hyperpigmentation (PIH) and keloidal scarring which are more prevalent in darker skin.

Recently, a fixed-dose combination product for the treatment of acne became available and contains a retinoid (adapalene) in combination with an antimicrobial (benzoyl peroxide [BPO]). Retinoids such as adapalene may be uniquely suited because they target key factors in hyperkeratinization and comedogenesis, and are anti-inflammatory and antifibroblastic. Adapalene itself possesses anticomedogenic, comedolytic, and anti-inflammatory properties.

Antimicrobials such as BPO provide additional benefits. BPO is an oxidizing agent with antibacterial and keratolytic effects and is used in acne treatment for its activities in decreasing the bacterial population of P. acnes. Thus far, no microbial resistance to BPO has been described. In addition, the nonclinical and clinical safety profile of BPO is well established.

Despite the benefits of combination therapy, the potential for increased cutaneous irritation is a concern. Although it has not been established that skin of color is more or less sensitive to irritants, PIH may be triggered in darker-skinned patients by skin irritation independent of cause, i.e. disease or iatrogenic cause. This problem has led some physicians to believe that skin of color is more sensitive to irritation from therapy. Because acne-related PIH is caused by a reaction to skin inflammation, minimizing inflammation and reducing potential irritation and dryness is also a key goal in treating acne in skin of color. This is a need for products that have minimal potential for irritation, while still being effective against acne, and are striving for a balance between early intervention and irritation potential of treatments to increase tolerability when treating subjects with skin of color.

The healing of a wound is a natural biological phenomenon which makes it possible, by repair and regeneration processes, to repair lesions.

The speed and the quality of the healing of a wound depend on the general condition of the organism affected, on the aetiology of the wound, on the condition and location of the wound, and on the occurrence or non-occurrence of an infection, and also on the genetic factors causing or not causing a predisposition to disorders of healing. Healing is the process which results in a scar. This process is also known as connective (or fibrous) organization of the inflammatory focus. The inflammatory reaction, by cellular and humoral mechanisms, induces the formation of an inflammatory granuloma which is gradually transformed into a regeneration blastema (or fleshy granulation) which constitutes the first stage of healing. The fleshy granulation is a transient newly formed connective tissue which will undergo significant modifications which ensure its transformation into a cicatricial fibrous tissue.

Inflammation is a dynamic process composed of a combination of vascular, cellular and humoral reactions triggered by any tissue lesion, whatever the cause (infectious, physical, chemical or ischemic). It makes possible the removal of the aggressive agent and cell debris and the repair of damaged tissue.

The healing process takes place in four main phases:
the initial vascular/exudative phase, which comprises active congestion of the vessels, an oedema and the migration of the leukocytes towards the site of the inflammation
the phase of forming the inflammatory granuloma, which is converted into a regeneration blastema, also known as fleshy granulation
the phase of cleaning (that is to say, the removal of the necrotic tissues, microorganisms, possible foreign bodies and the oedema fluid), of inflammation and of epithelialization (that is to say, multiplication of the epidermal cells and end of healing)
the healing phase proper, which makes possible the change from a fleshy granulation to the cicatricial fibrous tissue (or scar).

Usually, a wound is healed after 10 days. Starting from the 60th day, the scar passes through a physiological hypertrophic phase, during which phase it will thicken and become connective and the neighbouring tissues will become retractile. This hypertrophic phase is virtually complete after 1 year. Afterwards, the scar is no longer red or stiff and does not cause pain; it becomes flat.

However, in some cases, healing does not take place as well and pathological scars are formed. The term “healing disorders” is then used. These disorders are conventionally defined as disruptions of healing; they bring together two phenomena:
ulcers, which are an abnormality of healing where the wound becomes hollow and where the granulation tissue is not reconstructed. Hypotrophic or atrophic scars, resulting in particular from traumas but also from skin pathologies, such as acne vulgaris or chicken-pox, are hollow areas or ice-pick scars; their form is also due to an abnormality of healing
hypertrophic scars and keloids (or “keloidal scars”), which are processes where the granulation tissue hyperproliferates in an abnormal fashion (Treatment of scars: a review, Alster et al., Ann. Plast. Surg., 1997, October, 39(4), 418-32).

Healing disorders thus bring together pathologies which are very different from the normal healing process.

The present invention is concerned with 2 types of pathological scars: “hypertrophic” scars and “keloid” or “keloidal” scars.

Whether hypertrophic or keloidal, these scars have as common origin an initial hyperplasic phase of high intensity and/or lengthy duration, which phase brings about an excess of dense fibrous tissue in the dermis. Pathological scars are large, swollen, red and hard, and itch.

The change in these scars over time makes it possible to distinguish a hypertrophic scar from a keloidal scar.

This is because:
hypertrophic scars spontaneously improve over time (in 2 or 3 years on average). They remain confined to the original site of the scar;
keloidal scars for their part do not have any tendency to spontaneously improve and remain stable, indeed even become worse, with time. Furthermore, this type of scar expands beyond the original site of the scar and affects the neighbouring healthy tissues.

The cause or causes at the root of the formation of these pathological scars are still poorly known but there are a number of factors which favour their onset.

Mention may be made, among the risk factors for the formation of pathological scars, of:
race: persons of the black or Asiatic race are much more subject to keloids than persons of the white race;
age: frequent in children, hypertrophic scars are rare in elderly subjects;
location on the body: some parts of the body are more prone to develop pathological scars, such as, for example, the sternum, neck, ear lobes or lower part of the face.

Intralesional excision or resection treatments for keloids in particular (in order not to again induce a lesion) exist in order to treat these pathological scars.

The treatment of hypertrophic and keloid scars is obviously not only surgical. As the cause of the hypertrophic scar is unknown, risks of recurrence after a simple surgical alteration to the scar exist. Surgery can certainly reduce the size of the scar when it is too large but it is then necessary to follow it, as rapidly as possible, with the following two methods, alone or in combination:
“pressure therapy”, carried out with made-to-measure compressive elastic clothing or also with silicone dressings with compression. It is highly effective, provided that it is applied permanently (day and night) for approximately 6 months, which is not always achievable;
“corticotherapy”, by injection inside the scar of prolonged-effect cortisone products. Due to the normal great hardness of these scars, the best method for injecting the product under pressure into the scar is to use a needleless device (“Dermo Jet”).

Treatments using interferon also exist but, currently, whatever the treatment used, complete disappearance of the lesions is only rarely achieved.

Post-inflammatory hyperpigmentation (PIH) causes skin darkening and discoloration that show up as spots, or as large patches on a person's body. This is because cells that normally produce brown pigment evenly across your skin go into overdrive and produce too much melanin. This happens because of an inflammatory reaction in, or to an injury to, the skin. If the excess melanin is produced in the upper layer of skin (epidermis), the pigmentation color is a darker shade of brown. If the excess melanin is produced in the lower layer of skin (the dermis), a gray or blue discoloration becomes visible.

Although PIH can occur in all skin types, it is more common in people of Africa, Asia, Latin, and indigenous Indian background, and can affect men and women equally. Areas of the skin affected by PIH correspond with areas of previous inflammation or injury. When dark changes in your skin's color remain after the underlying problem has gone away, you have PIH. The most common causes are injuries such as scratches, burns, cuts, or bruises. Rashes of any type can cause PIH (examples of which include eczema, psoriasis, pityriasis rosea, lichen planus, and fungal infections). Ordinary conditions such as acne or pimples are a very common cause of PIH in individuals with brown skin. PIH can also be caused by injury to the skin resulting from sunburns, surgery or cosmetic procedures such as chemical peels, dermabrasion, lasers and cryotherapy (liquid nitrogen treatments).

It is also important to know that PIH will in many cases fade over time on its own. However, there are treatments available that can speed up the process.

For patients with PIH, the most common way to return the skin to its natural glowing complexion is through use of products containing Hydroquinone, a chemical lightening agent that is applied directly to the dark mark. Your dermatologist can determine if hydroquinone is appropriate for your skin. Many dermatologists consider hydroquinone to be the best treatment for PIH. Hydroquinone works by blocking an enzyme that is responsible for the production of the pigment melanin. By blocking the formation of melanin, the dark area will lighten. However, it is important to realize that you may need to use the hydroquinone medication for up to 6 months before clearing of the dark marks is seen.

For the treatment of PIH, Retin-A has been studied in individuals with brown skin. For these patients with acne and PIH, the retinoid is applied to the acne and PIH prone areas nightly.

Azelaic acid is another prescription treatment for both acne and PIH. It has anti-inflammatory, anti-bacterial, and skin lightening properties. A small amount of this cream is applied to the acne and PIH prone skin once or twice daily. It is particularly useful for individuals who are unable to tolerate the hydroquinone products. Improvement in the dark marks can be seen after 6 months.

Glycolic acid products are available over the counter and are also used as a treatment for PIH. These products work by gently exfoliating (removing) the upper-most layer of the skin and the dark marks with it. There are many products that contain glycolic acid. These include cleansers, lotions, gels, toners and creams. The concentration of glycolic acid contained in the products range from 5 to 20 percent

Acne vulgaris is common among individuals of all ethnic skin types, its specific prevalence based on race has not been determined. However, it is known that darker-skinned patients have a higher incidence of postinflammatory hyperpigmentation (PIH) and keloidal scarring. Also, it has been found that Hispanic patients present more frequently with papular lesions, acne hyperpigmented macules (AHM) and cystic lesions (74.5%, 52.7% and 25.5%, respectively).

Therefore, in order to prevent these damaging sequelae, the need to treat non-caucasian patients with acne early and effectively is essential. The adapalene-BPO combination has been shown to provide significant benefit relative to its monotherapies and vehicle in reduction of lesion counts as early as week 1, with an acceptable tolerability profile.

Surprisingly, the inventors have discovered that the combination of Adapalene and benzoyl peroxide makes it possible to efficiently treat acne and particularly reducing the incidence of post inflammatory hyperpigmentation (PIH) due to lesions and thus to improve the flexibility of pathological scars and hyperpigmented scars.

The present invention shows through an analysis among Hispanic patients with acne that adapalene-BPO acts quickly and effectively on reducing lesions (particularly inflammatory) with a good safety profile, which is particularly important in this population at high risk for PIH and scarring.

A subject-matter of the present invention is thus the use of Adapalene and benzoyl peroxide for the preparation of a medicament which is intended for the treatment of acne in non-Caucasian population preferably skin colored patient population such as Hispanic, asian or darker-skinned patients. In a preferred embodiment, such combination of Adapalene and benzoyl peroxide decrease or prevent or avoid at least one sign or symptome of pathologies or disorders selected from post-inflammatory hyperpigmentation, pathological scars en preferentially keloid scarring, acne hyperpigmented macules or cystic lesions.

According to the invention, the term “pathological scars” is understood to mean hypertrophic scars and keloidal scars.

Advantageously, the medicament according to the present invention is intended for topical application.

The medicament according to the present invention also comprises a physiologically acceptable medium, that is to say a medium which is compatible with the skin, including the scalp, mucous membranes, hair, body hairs and/or eyes, and can constitute a dermatological composition.

The present invention regards to the compound 6-[3-(1-Adamantyl)-4-methoxyphenyl]-2-naphthoic acid (referred to hereinbelow as adapalene) is a naphthoic acid derivative with retinoid and anti-inflammatory properties as well as its salts.

Adapalene is sold under the brand name Differin® at a weight concentration of 0.1%, in the form of an “alcoholic lotion” solution, an aqueous gel and a cream. These compositions are intended for treating acne. Patent application FR 2 837 101 describes adapalene compositions at a weight concentration of 0.3%, for treating acne.

Patent application WO 03/055 472 moreover describes stable pharmaceutical compositions comprising adapalene and benzoyl peroxide (BPO).

The term “adapalene salts” means the salts formed with a pharmaceutically acceptable base, especially mineral bases such as sodium hydroxide, potassium hydroxide and ammonia or organic bases such as lysine, arginine or N-methylglucamine. The term “adapalene salts” also means the salts formed with fatty amines such as dioctylamine and stearylamine.

The expression “combination of adapalene or salts thereof with benzoyl peroxide” means a single composition comprising both adapalene or salts thereof and benzoyl peroxide.

Effectiveness of benzoyl peroxide is due to decomposition when in contact with skin. Indeed, these are the properties of oxidizing free radicals produced during the decomposition leading to the desired effect. Also, to maintain the benzoyl peroxide optimum efficiency, it is important to prevent decomposition before use, ie during storage.

But benzoyl peroxide is an unstable chemical compound that makes it difficult to formulate into finished products. The solubility and stability of benzoyl peroxide have been studied by Chellquist et al. in ethanol, propylene glycol and various mixtures of polyethylene glycol 400 (PEG 400) and water (Chellquist E M and W G Gorman, Pharm. Res., 1992, Vol 9: 1341-1346).

Preferably, the composition of the invention comprises between 0.001% and 5% and preferably between 0.01 and 1% by weight of adapalene relative to the total weight of the composition, preferably between 0.01% and 0.5%, preferably between 0.1% and 0.4% by weight of adapalene, even more preferably 0.1% or 0.3% by weight of adapalene.

The composition includes also benzoyl peroxide (BPO). In the compositions of the invention, the benzoyl peroxide is used at concentrations ranging from 1% to 10% by weight, more particularly from 2% to 7% by weight, more preferably from 2.5% to 5% weight relative to the total weight of the composition.

Benzoyl peroxide can also be used in the free form or in an encapsulated form adsorbed on or absorbed in any porous medium. It may be, for example—benzoyl peroxide encapsulated in a polymer system consisting of porous microspheres, for example MISCROSPONGED sold under the name Microsponges P009A Benzoyl peroxide by the company Cardinal Health.

All the pharmaceutical compositions that are useful in the invention may comprise from 0.01% to 2%, preferably between 0.05% and 0.5% and preferentially between 0.1% and 0.3% of adapalene, and from 0.1% to 20% and preferably from 0.5% to 10% of BPO, more preferably from 2% to 5% of BPO and preferentially 2.5% of BPO.

All the percentages are indicated by weight relative to the total weight of the composition.

Furthermore, the composition as described above can comprise all the constituents normally present in the type of application envisaged.

The medicament according to the present invention can comprise a large variety of additional components; in particular, they can be absorbents, abrasives, antiacne agents, antifoaming agents, antimicrobial agents, antioxidants, binders, biological additives, buffers, chelating agents, colorants, cosmetic astringents, cosmetic biocides, external analgesics, film-forming agents, fragrance components, opacifying agents, plasticizers, preservatives, other depigmenting agents, emollients, skin-protecting agents, solvents, solubilizing agents, surfactants, agents which absorb ultraviolet light, sunscreens, viscosity-increasing agents (aqueous or nonaqueous), humectants, sequestering agents, and the like.

These additional components can be present in the medicament according to the present invention in an amount of between 0.001% and 20% by weight, with respect to the total weight of the medicament.

A person skilled in the art will obviously take care to choose the possible additional compounds and/or their amounts so that the advantageous properties of the medicament according to the present invention are not completely or not substantially reduced by the envisaged addition.

The medicament of the present invention is a composition which can be in any pharmaceutical form normally used for topical application, such as aqueous dispersions, aqueous or oily suspensions, aqueous gels, anhydrous or lipophilic emulsions (lotions, creams or ointments) of liquid, semisolid or solid, obtained by dispersing a fatty phase in an aqueous phase (O/W) or conversely (W/H) in the presence or absence of emulsifier, or micro emulsions, micro capsules, micro particles or vesicular dispersions of ionic and/or nonionic.

Thus in preferred embodiment, the medicament according to the present invention can be provided in any pharmaceutical dosage form normally used in the field of dermatology. Preferably, the medicament will be provided in the emulsion (lotions, creams, cream without emulsifier), suspensions or gel form.

Furthermore, they can comprise other normal ingredients of creams and be manufactured in a way well known to a person skilled in the art.

Advantageously, the medicament according to the present invention comprises at least one inactive ingredient chosen from butylated hydroxytoluene, cetyl alcohol, citric acid, glycerol, glyceryl stearate, magnesium aluminium silicate, methyl gluteth-10, methylparaben, PEG-100 stearate, propylparaben, purified water, sodium metabisulphite, stearic acid and stearyl alcohol.

Advantageously, the medicament according to the present invention corresponds to the aqueous gel Epiduo® sold by Galderma, as presented in Example 1.

### FIGURES DESCRIPTION

FIG. 1 shows the Median % Change from Baseline (ITT-LOCF) for Post-Inflammatory Hyper-pigmentation.

FIG. 2 shows the Percent of patients who did not experience any sign/symptom of a) erythema, b) scaling, c) dryness and d) burning/stinging at Week 1, stratified by Fitzpatrick skin type (I-III versus IV-VI)

### EXAMPLES

### Example 1

### Composition of the Aqueous Gel Epiduo®

The gel has the following formulation, as percentage by weight with respect to the total weight:
2.5% of BPO;
0.1% of adapalene;
0.10% of disodium EDTA;
4.00% of glycerol;
4.00% of propylene glycol;
   and also, preferably:
0.05% of sodium docusate;
0.20% of poloxamer 124;
4.00% of sodium acryloyldimethyltaurate copolymer and isohexadecane and polysorbate 80;
NaOH, in an amount sufficient to obtain a pH of 5.

### Example 2

### Adapalene-Benzoyl Peroxide Combination Relative to Vehicle is Quickly Effective in the Treatment of Acne Among a Hispanic Population

The aim of this example is to demonstrate that Epiduo is very efficient for treating acne in the sub-group of darker-skinned (also denominate non-caucasian) patients who have a higher incidence of postinflammatory hyperpigmentation (PIH) and keloidal scarring. In particular, it has been found that Hispanic patients present more frequently with papular lesions, acne hyperpigmented macules (AHM) and cystic lesions (74.5%, 52.7% and 25.5%, respectively).

Therefore, in order to prevent these damaging sequelae, the need to treat Hispanic patients with acne early and effectively is essential. The adapalene-BPO combination has been shown to provide significant benefit relative to its monotherapies and vehicle in reduction of lesion counts as early as week 1, with an acceptable tolerability profile. This subgroup analysis aimed to evaluate the benefit of adapalene-BPO relative to vehicle among Hispanics.

### Materials and Methods:

Data were pooled from three randomized, double-blind, controlled studies (a total of 3855 subjects), in which the efficacy and safety of adapalene-BPO were compared to those of its vehicle over a 12-week daily treatment period. For each type of lesion (total, inflammatory and non-inflammatory), efficacy (% change of lesion counts from baseline) and IGA success rate (investigator's global assessment; success rate defined as the % of subjects rated “clear/almost clear”) were evaluated for the Hispanic subgroup. Safety was evaluated using tolerability scores and adverse events assessed at each study visit.

### Results and Conclusion:

The Hispanic subgroup included 215 subjects (112 in adapalene-BPO group and 103 in vehicle group), with the majority having phototype IV, moderate acne and less than 18 years old. For inflammatory lesions, adapalene-BPO was significantly more efficacious than vehicle as early as week 1 (P<0.05) and at all study visits (−58.1% vs. −32.5%, or an added reduction of 25.6% over vehicle at week 12, P<0.001), in terms of median percentage changes from baseline. At week 2, nearly half of this effect had already been obtained. After 12 weeks, adapalene-BPO also significantly reduced non-inflammatory and total lesion counts (with a benefit of 25.7% and 29.3%, respectively, P<0.001). Additionally, about a quarter of subjects in the adapalene-BPO group were rated “clear/almost clear” [17% higher success than vehicle (27.7% vs. 10.7%), P=0.003]. Whereas there were more treatment-related adverse events (AEs) for adapalene-BPO compared to vehicle (16.1% subjects vs. 6.8%, respectively), the most frequent being dry skin, these AEs were of mild to moderate severity and none led to study discontinuation. Transient irritation (mainly at week 1) occurred in some patients treated with adapalene-BPO, but all tolerability worst scores remained below 1 (mild).

This analysis among Hispanic patients with acne demonstrated that adapalene-BPO acts quickly and effectively on reducing lesions (particularly inflammatory) with a good safety profile, which is particularly important in this population at high risk for PIH and scarring.

### Example 3

### Post-Inflammatory Hyperpigmentation Assessment

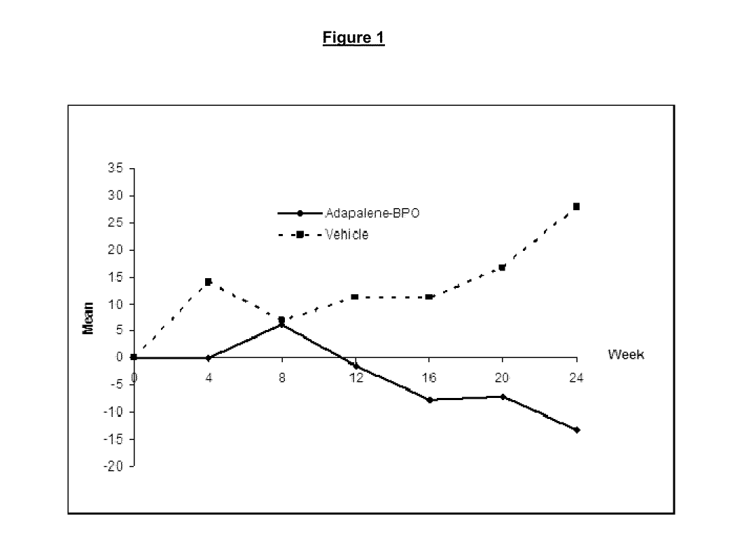

Post-inflammatory hyperpigmentation was assessed for 43 subjects eligible for this assessment, 24 for Adapalene-BPO group and 19 for Vehicle group. The analysis of Post-inflammatory hyperpigmentation assessment at each visit for the ITT population is shown in following table I and FIG. 1.

TABLE I
Post-Inflammatory Hyperpigmentation Assessment: % Change from
Baseline, Descriptive and p-value (ITT-LOCF)

| | | Adapalene-BPO | Vehicle | p-value |
|---|---|---|---|---|
| | | (n = 24) | (n = 19) | (1) |
| Week 4 | Mean ± STD | 0.00 ± 39.01 | 14.04 ± 53.94 | 0.366 |
| | (Min, Max) | (−50.00, 100.00) | (−50.00, 200.00) | |
| Week 8 | Mean ± STD | 6.25 ± 26.83 | 7.02 ± 55.92 | 0.254 |
| | (Min, Max) | (−50.00, 100.00) | (−50.00, 200.00) | |
| Week 12 | Mean ± STD | −1.39 ± 30.26 | 11.40 ± 59.85 | 0.965 |
| | (Min, Max) | (−50.00, 100.00) | (−50.00, 200.00) | |
| Week 16 | Mean ± STD | −7.64 ± 37.42 | 11.40 ± 59.85 | 0.534 |
| | (Min, Max) | (−100.0, 100.00) | (−50.00, 200.00) | |
| Week 20 | Mean ± STD | −6.94 ± 36.75 | 16.67 ± 63.10 | 0.420 |
| | (Min, Max) | (−100.0, 100.00) | (−50.00, 200.00) | |
| Week 24 | Mean ± STD | −13.19 ± 41.99 | 28.07 ± 61.12 | 0.020 |
| | (Min, Max) | (−100.0, 100.00) | (−33.33, 200.00) | |

(1) p-value for between treatment difference, by CMH test based on ridit scores

Again as shown in FIG. 1, Adapalene-BPO produced Post-inflammatory hyperpigmentation decrease from Week 12 compared to Vehicle. A statistically significant difference in favour of Adapalene-BPO group occurred at Week 24 (−13.19% versus +28.07%, p<0.020). Thus after 24 weeks, PIH tended to decrease with Epiduo® whereas it increased with vehicule (p<0.02).

### Example 4

### Investigation of Possible Difference Differences in the Incidence and Severity of Irritation Among Different Skin Type Classifications and Race or Ethnicity

A meta-analysis of the cutaneous irritation of the adapalene-BPO gel was conducted to investigate possible differences in the incidence and severity of irritation among different skin type classifications and race or ethnicity. Three randomized, double-blind, vehicle- and placebo-controlled clinical trials involving a total of 3,855 patients have established the safety and efficacy of adapalene-BPO gel in the treatment of acne for all skin types. The present post-hoc, meta-analysis is based on the tolerance data from those patients who were assigned to the adapalene 0.1%-BPO 2.5% treatment arm in each of the 3 randomized trials.

### Methods

All 3 studies had similar objectives and design. They were multicenter, randomized, double-blind, parallel-group, active- and vehicle-controlled studies. Study 1 was conducted at 60 centers in the United States, Puerto Rico, and Canada. Study 2 was conducted at 61 centers in the United States, Canada and Europe, and study 3 was conducted at 36 centers in the United States. The efficacy and safety of the adapalene 0.1%-BPO 2.5% combination gel was compared with adapalene and BPO monotherapies as well as the gel vehicle. Participants were randomized to adapalene-BPO combination gel, adapalene gel monotherapy, BPO gel monotherapy, or gel vehicle. Efficacy and safety evaluations were performed at baseline and weeks 1, 2, 4, 8, and 12 and included investigator ratings for erythema, scaling, dryness, and stinging/burning on a scale ranging from 0 (none) to 3 (severe).

Patients enrolled in the 3 studies were male or female of any race, 12 years of age or older with facial lesions counts (excluding the nose) between 20 and 50 for inflammatory lesions and between 30 and 100 for non-inflammatory lesions, no cysts and no more than 1 nodule in studies 1 and 2 (no cysts or nodules in study 3).

All studies included in this meta-analysis were conducted in accordance with the ethical principles originating from the Declaration of Helsinki and Good Clinical Practices and in compliance with local regulatory requirements. The studies were reviewed and approved by an institutional review board or ethics committee. Prior to the performance of any study procedures, written informed consent was obtained from all participants.

### Statistical Analysis

This meta-analysis included subjects who were randomized to the Adapalene-BPO treatment group in each of the 3 studies. In all 3 studies, it was determined that week 1 represented the worst severity of irritation, so only week 1 assessments were included in this meta-analysis. For each study, and for the combined-study meta-analysis, comparisons were performed among the following subgroups: 1) subjects with Fitzpatrick skin types I-III versus subjects with Fitzpatrick skin types IV-VI; 2) Caucasian subjects versus non-Caucasian subjects; 3) Caucasian subjects versus Black subjects versus Hispanic subjects (3-way analysis).

Each tolerability score for erythema, scaling, dryness, and stinging/burning, was treated as a categorical variable. Cochran-Mantel-Haenszel (CMH) tests were used to determine statistically significant differences between sub-populations, controlling for study site in the analysis of individual studies and for study site and study number in the combined-study meta-analysis.

Additionally, a sensitivity analysis was performed using the CMH test to investigate whether combining the three clinical studies had introduced a bias. To investigate differences between the groups of patients treated in each study and in the combined-study analysis, the CMH test controlling for potentially confounding variables was used to test relationships (if any) among the four groups.

All tests were two sided and used the 0.05 level to declare significance. No adjustment for multiplicity was made.

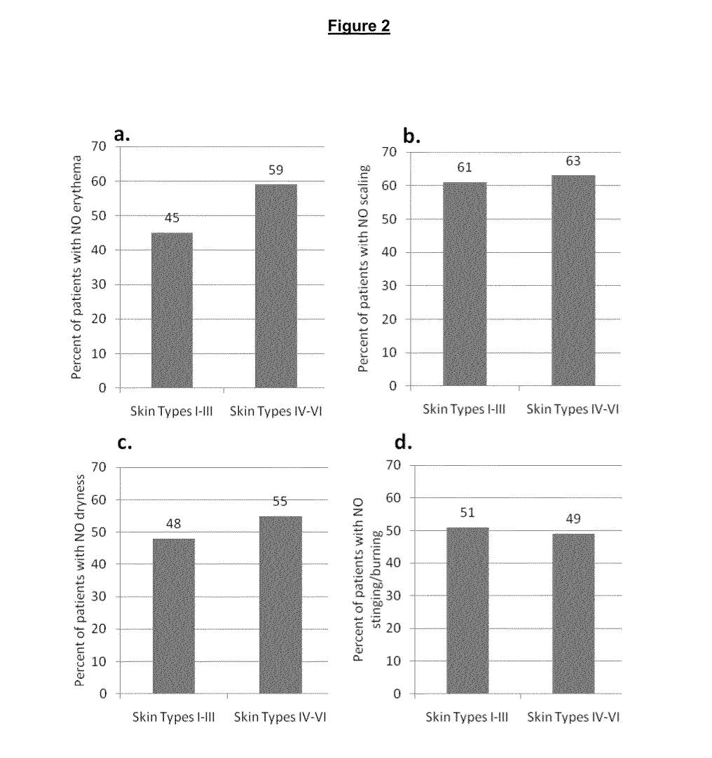

### Results (FIG. 2)

A total of 983 patients received at least 1 dose of adapalene-BPO in the 3 studies. The meta-analysis population includes 909 patients, as 74 patients (7.5%) who did not return for week 1 visit (eg, because they were lost to follow-up or withdrew for personal reason, treatment failure, of the occurrence of adverse events) were not included in the analysis. The majority of patients were Caucasian (73%), 11% were Black, and 11% were Hispanic Fitzpatrick skin types I-III described 73% of the patients, while 37% had types IV-VI.

There were not statistically significant differences in the demographics variables among the 3 studies, thus allowing to combine the studies into a meta-analysis. When general linear models were used to model the data, with tolerability assessments used as dependent variables and other demographics variables and study number used as independent variables, the coefficients for race and Fitzpatrick Skin Type were not significant (P=NS).

### Cutaneous Irritation Scores by Fitzpatrick Skin Type

The relationship between each of the 4 cutaneous irritation scores and Fitzpatick skin type is shown in for each individual study and for the combined-study analysis. In each of the 4 categories, 45% of the patients or more did not experience the sign/symptom of irritation (FIG. 2). Among those patients who experienced irritation, the reports were mostly mild; they occurred early and resolved while still on treatment (not shown).

A statistically significant difference in the distribution of erythema severity among subjects with Fitzpatrick skin types I-III compared to subjects with Fitzpatrick skin types IV-VI was noted in the combined-study analysis (P<0.001), with more patients with Fitzpatrick skin types IV-VI reporting a score of “none” for erythema (59% versus 45%) and fewer of them reported erythema rated as mild (30% versus 40%). The same statistically significant difference was noted in study 1 (P<0.001) although not in study 2 or 3.

There were no statistical differences in the distribution of scaling, dryness, or stinging/burning in any of the 3 individual studies or in the combined-study meta-analysis when subjects with Fitzpatrick skin types I-III were compared to subjects with Fitzpatrick skin types IV-VI. The only exception was for dryness in study 3 (P=0.017).

### Cutaneous Irritation Scores by Ethnicity

The relationship between each of the 4 cutaneous irritation scores and race or ethnicity is shown in for the combined-study analysis. Significant differences between racial or ethnic groups were found in the distribution of all 4 parameters.

When Caucasian subjects were compared to non-Caucasian subjects, more non-Caucasian patients reported a score of “none” for erythema (63% versus 45%) and fewer of them reported erythema rated as mild (29% versus 39) or moderate (7% versus 15%) (P<0.001). This statistically significant difference was noted in study 1 (P<0.001) but not in studies 2 or 3 (not shown). When Caucasians were compared to Blacks and to Hispanic in a 3-way comparison, the combined-study meta-analysis also revealed a statistically significant difference with fewer mild events and more “none” ratings for erythema in Black patients compared to Caucasians or Hispanic patients (P<0.001).

Combined results from the 3 studies also revealed a statistically significant difference in the distribution of scaling severity with the Caucasian group having fewer patients with a rating of “none” for scaling and more patients with mild scaling that non-Caucasian subjects (P<0.001). No statistically significant differences were noted in any of the 3 studies individually (not shown). When Caucasians were compared to Blacks and to Hispanic, the combined-study meta-analysis revealed a statistically significant difference with the Black sub-group having more patients with “none” ratings for scaling and fewer patients with mild scaling than the other 2 sub-groups (P=0.005).

For dryness, the Caucasian subjects had fewer patients with a rating of “none” for dryness and more patients with mild dryness that the non-Caucasian subjects (P<0.001 for the combined-study analysis). This statistically significant difference was noted in study 1 (P<0.001) but not in studies 2 or 3 (not shown). When results were compared among Caucasians versus Blacks versus Hispanic, the Caucasian group had fewer patients with “none” ratings for dryness and more patients with mild dryness that either the Black group or the Hispanic group (P<0.001 for the combined-study meta-analysis).

There were no statistically significant differences in the distribution of stinging/burning severity among Caucasian subjects compared to non-Caucasian subjects in any of the 3 studies or for the 3 studies combined. When results were compared between Caucasians, Blacks and Hispanic, the combined-study analysis revealed a statistically significant difference with Black subjects having less mild and more moderate burning/stinging (P=0.003).

### CONCLUSIONS

This meta-analysis of 3 randomized clinical trials was conducted to determine if tolerability of adapalene-BPO gel treatment was different in subjects with different Fitzpatrick skin type or race. Contrary to the belief that subjects with skin of color are more sensitive to many topical acne treatments than Caucasians, the results of this meta-analysis showed no statistically significant differences in dryness, scaling, and stinging/burning with adapalene-BPO treatment when subjects with Fitzpatrick skin types I-III were compared to subjects with skin types IV-VI (P=NS). Only erythema assessments were statistically different based on Fitzpatrick skin types with the lighter skin types faring worse than the darker skin types (P<0.001). Although this latter finding may be explained in part by the fact that mild to moderate erythema would be less visible on darker skin, the results suggest that the adapalene-BPO formulation is not associated with higher incidence of any signs or symptoms of irritation in higher Fitzpatrick skin types. When results were stratified further by race or ethnicity, non-Caucasians were scored “none” more frequently and “mild” or “moderate” less frequently than Caucasians with respect to erythema, dryness, and scaling (P<0.001); there was no significant differences in stinging/burning (P=0.571). Blacks and Hispanics were scored as “none” significantly more than Caucasians in each one of the 4 signs or symptoms (P 0.005).

1. A method of preparing a medicament for treating acne in a non-Caucasian subject, the method comprising preparing the medicament by combining effective amounts of Adapalene and benzoyl peroxide.

2. The method as defined by claim 1, wherein administration of the medicament decreases at least one sign or symptom of pathologies or disorders selected from the group consisting of post-inflammatory hyperpigmentation, keloid scarring, acne hyperpigmented macules and cystic lesions.

3. The method as defined by claim 1, wherein the medicament is in a form suitable for topical application.

4. The method as defined by claim 1, wherein the benzoyl peroxide is present at a concentration of from 1% to 10% by weight with respect to the total weight of the medicament.

5. The method as defined by claim 1, wherein the Adapalene is present at a concentration of from 0.001% to 20% by weight, with respect to the total weight of the medicament.

6. The method as defined by claim 1, wherein the medicament is in the form of a gel, emulsion, or lotion.

7. The method as defined by claim 1, wherein the medicament is in the form of a composition comprising an aqueous gel having the following composition, as percentage by weight with respect to the total weight:
2.5% of BPO;
0.1% of adapalene;
0.10% of disodium EDTA;
4.00% of glycerol;
4.00% of propylene glycol;
and also, optionally:
0.05% of sodium docusate;
0.20% of poloxamer 124; and
4.00% of sodium acryloyldimethyltaurate copolymer and isohexadecane and polysorbate 80.

8. The method as defined by claim 4, wherein the benzoyl peroxide is present at a concentration of from 2% to 5% by weight.

9. The method as defined by claim 4, wherein the benzoyl peroxide is present at a concentration of 2.5% by weight.

10. The method as defined by claim 5, wherein the Adapalene is present at a concentration of from 0.005% to 1% by weight.

11. The method as defined by claim 5, wherein the Adapalene is present at a concentration of from 0.1% to 0.3% by weight.

12. A method of treating acne in a non-Caucasian subject in need thereof, the method comprising administring a medicament to the non-Caucasian subject wherein the medicament comprises an effective amount of a combination of Adapalene and benzoyl peroxide.

13. The method as defined by claim 12, wherein administration of the medicament decreases at least one sign or symptom of pathologies or disorders selected from the group consisting of post-inflammatory hyperpigmentation, keloid scarring, acne hyperpigmented macules and cystic lesions.

14. The method as defined by claim 12, wherein the medicament is in a form suitable for topical application.

15. The method as defined by claim 12, wherein the benzoyl peroxide is present at a concentration of from 1% to 10% by weight, with respect to the total weight of the medicament.

16. The method as defined by claim 15, wherein the benzoyl peroxide is present at a concentration of from 2% to 5% by weight.

17. The method as defined by claim 15, wherein the benzoyl peroxide is present at a concentration of 2.5% by weight.

18. The method as defined by claim 12, wherein the Adapalene is present at a concentration of from 0.001% to 20% by weight, with respect to the total weight of the medicament.

19. The method as defined by claim 18, wherein the Adapalene is present at a concentration of from 0.005% to 1% by weight.

20. The method as defined by claim 18, wherein Adapalene is present at a concentration of from 0.1% to 0.3% by weight.

21. The method as defined by claim 12, wherein the medicament is in the form of a gel, emulsion, or lotion.

22. The method as defined by claim 12, wherein the medicament is in the form of a composition comprising an aqueous gel having the following composition, as percentage by weight with respect to the total weight:
2.5% of BPO;
0.1% of adapalene;
0.10% of disodium EDTA;
4.00% of glycerol;
4.00% of propylene glycol;
and also, optionally:
0.05% of sodium docusate;
0.20% of poloxamer 124; and
4.00% of sodium acryloyldimethyltaurate copolymer and isohexadecane and polysorbate 80.
